(19) United States
(12) Patent Application Publication
(10) Pub. No.: US 2021/0113181 A1
(43) Pub. Date: Apr. 22, 2021

(54) Automatic Ultrasonic Scanning System

(71) Applicant: Zhejiang Demetics Medical Technology Co., Ltd., Hangzhou (CN)

(72) Inventors: Fa Wu, Hangzhou (CN); Siyuan Jiang, Hangzhou (CN); Chaowei Chen, Hangzhou (CN); Wei Zheng, Hangzhou (CN)

(21) Appl. No.: 16/994,185

(22) Filed: Aug. 14, 2020

Related U.S. Application Data
(63) Continuation of application No. PCT/CN2019/117108, filed on Nov. 11, 2019.

(30) Foreign Application Priority Data
Oct. 22, 2019 (CN) 201911003898.7
Nov. 6, 2019 (CN) 201911073609.0

(51) Int. Cl. A61B 8/00; B25J 9/16; B25J 13/08

(52) U.S. Cl. CPC A61B 8/4218; A61B 8/56; A61B 8/4444; A61B 8/54; B25J 9/1689; A61B 2090/064; B25J 9/1633; B25J 13/08; B25J 9/1697; B25J 9/1684; B25J 9/1664; B25J 13/085

(57) ABSTRACT

An automatic ultrasonic scanning system includes a robotic arm with a camera, an ultrasonic probe mounted at an end of the robotic arm, a six-dimension force sensor, and a host computer. The six-dimension force sensor is fixed at the end of the robotic arm, and the ultrasonic probe is fixed on the six-dimension force sensor via a clamp. The six-dimension force sensor can detect a reactive force generated when the ultrasonic probe is in contact with a body surface of a person. The host computer is connected with each of the six-dimension force sensor, the camera and an image collection card via a data line. A controller of the robotic arm is connected to the host computer via an Ethernet communication bus. The ultrasonic machine is connected to the image collection card via a data line.

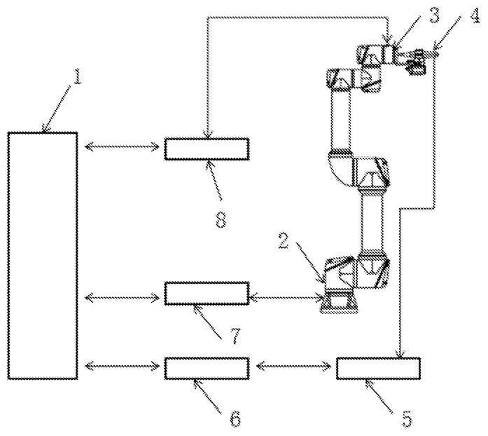

### CROSS-REFERENCE TO RELATED APPLICATIONS

This application is a continuation application of PCT patent application No. PCT/CN2019/117108 filed on Nov. 11, 2019, which claims priorities of Chinese Patent Application No. 201911003898.7 filed on Oct. 22, 2019 and Chinese Patent Application No. 201911073609.0 filed on Nov. 6, 2019. The entire contents of the above-identified applications are incorporated herein by reference.

### FIELD OF THE INVENTION

The present disclosure relates to the technical field of medical instruments, and in particular, to an automatic ultrasonic scanning system having a force sensor. A method for using the automatic ultrasonic scanning system is further provided.

### BACKGROUND OF THE INVENTION

Ultrasonic scanning is a relatively cheap and effective way for body surface scanning and detection, and it is of great help to preliminary positioning and estimating of lesions. In recent years, as people pay more attention to physical health, there is an increasing demand for ultrasound scanning.

However, current ultrasound scanning also has shortcomings. Firstly, for doctors, ultrasound scanning is a repetitive and tedious job, and working in a specific posture for a long time can lead to diseases such as arthritis. Secondly, for remote areas with underdeveloped medical treatment, there is a lack of experienced doctors (or even a lack of professional doctors) to complete a scanning operation process. Finally, due to differences in the personal professional quality of doctors, ultrasound images cannot be standardized, and acquired image information cannot be widely recognized.

At present, there are various devices that can automatically perform ultrasonic scanning, but these devices have a problem of lack of tactile control. As a result, an ultrasonic probe at an end of a robotic arm cannot control an intensity of a contact force in real time during a scanning process, and this may even cause discomfort because of excessive pressure on a body surface of a patient, so that usage experience is not good. Scanning techniques of experienced professional doctors cannot be simulated certainly. Therefore, it is necessary to provide a new automatic ultrasonic scanning system to solve the above-mentioned shortcomings in the existing technologies.

### SUMMARY OF THE INVENTION

The technical problem to be solved by the present disclosure is to provide an automatic ultrasonic scanning system so as to overcome shortcomings in the existing technologies.

In order to solve the technical problems, the present disclosure adopts the following solutions.

Provided is an automatic ultrasonic scanning system, including a robotic arm with a camera and an ultrasonic probe mounted at an end of the robotic arm. The ultrasonic probe is connected to an ultrasonic machine via a signal line. The system further includes a six-dimension force sensor and a host computer. The six-dimension force sensor is fixed at the end of the robotic arm, and the ultrasonic probe is fixed on the six-dimension force sensor via a clamp. The six-dimension force sensor is capable of detecting a reactive force generated when the ultrasonic probe is in contact with a body surface of a person. The host computer is connected with each of the six-dimension force sensor, the camera and an image collection card via a data line, and a controller of the robotic arm is connected to the host computer via an Ethernet communication bus. The ultrasonic machine is connected to the image collection card via a data line.

In the present disclosure, the host computer is connected with each of the six-dimension force sensor, the camera and the image collection card via a USB transmission line, and the ultrasonic machine is connected to the image collection card via an HDMI line.

In the present disclosure, the camera is a depth camera or a contour camera.

Provided further in the present disclosure is a method for using the above-mentioned automatic ultrasonic scanning system. The method includes the following steps of:

(1) allowing a patient to be detected to lie on his/her back, and acquiring, by the camera in combination with hand-eye calibration of the robotic arm, spatial curved surface information of a scanning position;

(2) selecting manually a start point of scanning and a desired scanning trajectory on the host computer according to scanning needs, and analyzing and acquiring, by the host computer, a position of a discrete point on the trajectory in a coordinate system of the robotic arm and a tangent vector and a normal vector of the discrete point;

(3) sending, by the host computer, an action command to the controller of the robotic arm, so that the robotic arm performs scanning to a body of a patient along the desired scanning trajectory; and receiving, by the host computer, signal data from the force sensor in real time during the scanning, then comparing the signal data with a preset numerical value of a contact force, and adjusting an intensity of a force applied on the body of the patient by the robotic arm according to a difference between the signal data and the preset numerical value of the contact force, so as to maintain a contact force detected by the force sensor always within a preset range; and

(4) transmitting, by the ultrasonic probe, ultrasonic detection signals during the scanning to the ultrasonic machine; and converting, by the ultrasonic machine, the ultrasonic detection signals into ultrasonic images, and transmitting the ultrasonic images to the host computer through the image collection card.

In the present disclosure, step (3) further includes: performing, by the host computer, interpolation for a desired contact force at each of discrete points on the desired scanning trajectory according to a preset rule, so as to simulate a specific scanning technique; or setting, by the host computer, the contact force at each of discrete points on the desired scanning trajectory to have a designated numerical value according to a preset rule.

In the present disclosure, step (4) further includes: performing, by the host computer, interpolation for a desired ultrasonic brightness at each of discrete points on the desired scanning trajectory according to a preset rule, so as to distinguish different areas; or setting, by the host computer, the ultrasonic brightness at each of discrete points on the desired scanning trajectory to have a designated numerical value according to a preset rule.

Compared with the existing technologies, the present disclosure has the following beneficial effects.

1. In the present disclosure, a force sensor is disposed between an end surface of a robotic arm and an ultrasonic probe, so that a contact force can be detected in real time during a scanning process and that detected data can be used in adjusting an acting force of the robotic arm. Discomfort or even pain caused by excessive pressure on a body of a patient can be avoided while scanning requirements are met.

2. Based on detection of the contact force, the present disclosure can realize applying different contact forces on positions of specific discrete points on a scanning trajectory, so as to simulate scanning techniques of experienced professional doctors. Therefore, the present disclosure provides an application prospect of using a robot to carry out scanning with “expert techniques”.

3. In the present disclosure, an ultrasonic brightness at a discrete point can also be adjusted, so as to distinguish ultrasonic scanning areas in combination with differences in contact forces, thereby improving precision of a scanning result.

### BRIEF DESCRIPTION OF THE DRAWINGS

The accompanying drawing schematically shows an automatic ultrasonic scanning system according to the present disclosure.

In the accompanying drawing, components and corresponding reference numbers are as follows: host computer 1; robotic arm 2; six-dimension force sensor 3; ultrasonic probe 4; ultrasonic machine 5; image collection card 6; Ethernet communication bus 7; and USB transmission line 8.

### DETAILED DESCRIPTION OF THE EMBODIMENTS

It should be noted first that, in the present disclosure, subject matters sought to be protected in the automatic ultrasonic scanning system only involve improvements to hardware devices and connection relationships thereof. A six-dimension force sensor used in this system is a mature commercial product and is directly available from the market.

In the present disclosure, the hardware devices except the force sensor and connection manners thereof are all existing technologies, and a method for controlling and using them so as to realize basic functions thereof are technical contents mastered by a person skilled in the art. For example, during control of a robotic arm, a robotic arm position-pose interpolation algorithm, a robotic arm force-position control algorithm and a corresponding robotic arm motion control algorithm are often used. Interpolation for the robotic arm means that for a series of desired waypoints and time intervals, a relationship between displacement and time is created by parameterization in a Cartesian space or a joint space, so that a desired trajectory of the robotic arm passes through each waypoint or passes by each waypoint in certain mixed radiuses. The applicant thinks that, in the present disclosure, application of these technologies does not go beyond a technical level of a person skilled in the art. After carefully reading the application documents and accurately understanding the implementation principles and objective of the present disclosure, a person skilled in the art can definitely use mastered skills to carry out the present disclosure in combination with the existing technologies, and thus the specific contents of these technologies are not described herein in detail.

Besides, the accompanying drawing schematically shows various views of the present disclosure. Herein, in order to provide clear illustration, some details are enlarged, and some details may be omitted. Shapes of respective components and relative sizes and positional relationships therebetween are only exemplary.

As shown in the accompanying drawing, the automatic ultrasonic scanning system includes a robotic arm 2 with a camera and an ultrasonic probe 4 mounted at an end of the robotic arm 2. The ultrasonic probe 4 is connected to an ultrasonic machine 5 via a signal line. This system further includes a force sensor 3 and a host computer 1. The six-dimension force sensor 3 is fixed at the end of the robotic arm 2, and the ultrasonic probe 4 is fixed on the six-dimension force sensor 3 via a well-matched clamp. The six-dimension force sensor 3 is used for detecting a reactive force generated when the ultrasonic probe 4 is in contact with a body surface of a person. The six-dimension force sensor 3 is connected to the host computer 1 via a USB transmission line. The ultrasonic machine is connected to an image collection card 6 via an HDMI line, and the image collection card 6 is connected to the host computer 1 via a USB transmission line. A controller of the robotic arm 2 is connected to the host computer 1 via an Ethernet communication bus 7. The camera is a depth camera or a contour camera, and is connected to the host computer 1 via a USB transmission line.

An example of a method for using the system is as follows.

A doctor performs operations through the host computer. The contour camera or the depth camera on the robotic arm 1 is used to acquire spatial curved surface information of a scanning position on a patient, and a start point and a desired scanning trajectory are selected on the host computer 1. The host computer performs automatic analysis, so as to acquire a tangent vector and a normal vector of a discrete point on the trajectory. The host computer 1 controls the robotic arm 2 to move to a position of the planned start point, to allow the ultrasonic probe 4 to contact with the scanning position on the patient. Then, the robotic arm 1 performs scanning along the desired scanning trajectory until the robotic arm reaches a planned end point. During this process, the ultrasonic probe 4 coincides with a normal vector of the trajectory all the time, so as to acquire clinically significant ultrasonic images while ensuring safety of the patient.

A method for carrying out automatic ultrasonic scanning by using this system includes the following steps.

(1) A patient to be detected is allowed to lie on his/her back, and spatial curved surface information of a scanning position is acquired by the camera in combination with hand-eye calibration of the robotic arm 2.

(2) A start point of scanning and a desired scanning trajectory are selected manually on the host computer 1 according to scanning needs, and a position of a discrete point on the trajectory in a coordinate system of the robotic arm and a tangent vector and a normal vector of the discrete point are analyzed and acquired by the host computer 1.

(3) The host computer 1 sends an action command to a controller of the robotic arm 2, so that the robotic arm 2 perform scanning to a body of a patient along the desired scanning trajectory. During the scanning, the host computer 1 receives signal data from the force sensor 3 in real time, then compares the signal data with a preset numerical value of a contact force, and adjusts an intensity of a force applied on the body of the patient by the robotic arm 2 according to a difference between the signal data and the preset numerical value of the contact force, so as to maintain a contact force detected by the force sensor 3 always within a preset range.

Further, interpolation may be performed for a desired contact force at each of discrete points on the desired scanning trajectory by the host computer 1 according to a preset rule, so as to simulate a specific scanning technique; or the contact force at each of discrete points on the desired scanning trajectory is set to have a designated numerical value by the host computer 1 according to a preset rule.

(4) Ultrasonic detection signals during the scanning are transmitted to the ultrasonic machine 5 by the ultrasonic probe 4; and then the ultrasonic detection signals are converted into ultrasonic images by the ultrasonic machine 5, and the ultrasonic images are transmitted to the host computer 1 through the image collection card 6.

Further, interpolation may be performed for a desired ultrasonic brightness at each of discrete points on the desired scanning trajectory by the host computer 1 according to a preset rule, so as to distinguish different areas; or the ultrasonic brightness at each of discrete points on the desired scanning trajectory is set to have a designated numerical value by the host computer 1 according to a preset rule.

1. An automatic ultrasonic scanning system, comprising a robotic arm with a camera and an ultrasonic probe mounted at an end of the robotic arm, the ultrasonic probe being connected to an ultrasonic machine via a signal line, wherein the system further comprises a six-dimension force sensor and a host computer, wherein the six-dimension force sensor is fixed at the end of the robotic arm, and the ultrasonic probe is fixed on the six-dimension force sensor via a clamp, the six-dimension force sensor being capable of detecting a reactive force generated when the ultrasonic probe is in contact with a body surface of a person; the host computer is connected with each of the six-dimension force sensor, the camera and an image collection card via a data line, and a controller of the robotic arm is connected to the host computer via an Ethernet communication bus; and the ultrasonic machine is connected to the image collection card via a data line.

2. The automatic ultrasonic scanning system according to claim 1, wherein the host computer is connected with each of the six-dimension force sensor, the camera and the image collection card via a USB transmission line, and the ultrasonic machine is connected to the image collection card via an HDMI line.

3. The automatic ultrasonic scanning system according to claim 1, wherein the camera is a depth camera or a contour camera.

4. A method for using the automatic ultrasonic scanning system according to claim 1, wherein the method comprises the following steps of:
(1) allowing a patient to be detected to lie on his/her back, and acquiring, by the camera in combination with hand-eye calibration of the robotic arm, spatial curved surface information of a scanning position;
(2) selecting manually a start point of scanning and a desired scanning trajectory on the host computer according to scanning needs, and analyzing and acquiring, by the host computer, a position of a discrete point on the trajectory in a coordinate system of the robotic arm and a tangent vector and a normal vector of the discrete point;
(3) sending, by the host computer, an action command to the controller of the robotic arm, so that the robotic arm performs scanning to a body of a patient along the desired scanning trajectory; and receiving, by the host computer, signal data from the force sensor in real time during the scanning, then comparing the signal data with a preset numerical value of a contact force, and adjusting an intensity of a force applied on the body of the patient by the robotic arm according to a difference between the signal data and the preset numerical value of the contact force, so as to maintain a contact force detected by the force sensor always within a preset range; and
(4) transmitting, by the ultrasonic probe, ultrasonic detection signals during the scanning to the ultrasonic machine; and converting, by the ultrasonic machine, the ultrasonic detection signals into ultrasonic images, and transmitting the ultrasonic images to the host computer through the image collection card.

5. The method according to claim 4, wherein step (3) further comprises: performing, by the host computer, interpolation for a desired contact force at each of discrete points on the desired scanning trajectory according to a preset rule, so as to simulate a specific scanning technique; or setting, by the host computer, the contact force at each of discrete points on the desired scanning trajectory to have a designated numerical value according to a preset rule.

6. The method according to claim 4, wherein step (4) further comprises: performing, by the host computer, interpolation for a desired ultrasonic brightness at each of discrete points on the desired scanning trajectory according to a preset rule, so as to distinguish different areas; or setting, by the host computer, the ultrasonic brightness at each of discrete points on the desired scanning trajectory to have a designated numerical value according to a preset rule.
